(19) United States
(12) Patent Application Publication
(10) Pub. No.: US 2012/0123089 A1
(43) Pub. Date: May 17, 2012

(54) PREPARATIVE NON-LINEAR GRADIENT BASED CHROMATOGRAPHIC METHOD AND PURIFIED PRODUCTS THEREOF

(75) Inventors: Nitesh Dave, Bangalore (IN); Devesh Radhakrishnan, New Panvel (IN); Sundaresh Shankar, Bangalore (IN); Krishanachaitanya Gulla, Indore (IN); Harish Iyer, Bangalore (IN)

(73) Assignee: BIOCON LIMITED, Bangalore (IN)

(21) Appl. No.: 13/382,299

(22) PCT Filed: Jul. 8, 2010
(86) PCT No.: PCT/IN10/00459

§ 371 Date: Jan. 5, 2012

(30) Foreign Application Priority Data
Jul. 9, 2009 (IN) 1639/CHE/2009

(51) Int. Cl. C07K 14/62; C07K 1/18; C07K 1/20; C07K 1/16

(52) U.S. Cl. 530/305; 530/344

(57) ABSTRACT

The instant disclosure provides a method for purification of peptides by chromatographic techniques. The proposed methodology will help in addressing the problems associated in purifying biological protein products emerging from the evolving biotechnology industry.

### FIELD OF THE INVENTION

A method for the purification of proteins by preparative chromatography on ion exchange/Reverse phase chromatographic media is disclosed. The invention generally relates to chromatography, and, more particularly, to methods of non-linear gradient based preparative chromatographic separations of target and byproduct materials affording better resolutions for separation leading to higher purities of the target compound. Further a method for the purification of insulin analog or derivative by RPHPLC using non-linear gradient elution is also disclosed.

### BACKGROUND OF THE INVENTION

Biological protein products emerging from the evolving biotechnology industry present new challenges for purification processing through chromatography. Typically, these products are large and labile having molecular weights in the range of 10⁴ to 10⁶ daltons. Such products are purified from mixtures which often containing hundreds of contaminating species including cell debris, various solutes, nutrient components, DNA and other impurities. The concentration of the protein product in the harvest liquor is sometimes as low as 1 mg/l but usually is on the order of 100 mg/l. The presence of proteases in the process liquor and their labile nature often mandates that purification be conducted as quickly as possible.

Chromatography is a dynamic separation process, which relies on the distribution of components to be separated between two phases: a stationary (or binding) phase bed and a mobile (or carrier) phase. The mobile phase carries the components to be separated through a column packed with the stationary phase Chromatographic techniques include separation based on ion-exchange, hydrophobic interaction etc. In reversed phase chromatography (RPC) a molecule in solution binds to the hydrophobic surface or hydrophobic ligand of a chromatographic resin.

A number of different chromatographic procedures are applied to obtain the desired end result with respect to purity and yield. Reverse-phase chromatography is one of the most powerful methods of purification employed utilizing hydrophobic interactions as the main separation principle. Reverse phase liquid chromatography (“RP-LC”) and reverse phase high-performance liquid chromatography (“RP-HPLC”) are commonly used to purify molecules such as peptides and proteins, produced by either synthetic or recombinant methods. RP-LC and RP-HPLC methods can efficiently separate closely related impurities and have been used to purify many diverse molecules (Lee et al., “Preparative HPLC,” 8th Biotechnology Symposium, Pt. 1, 593-610 (1988)). Further, RP-LC and RP-HPLC have been successfully used to purify molecules, particularly; proteins on an industrial scale (Olsen et al., 1994, J. Chromatog. A, 675, 101).

The Ion exchange chromatography principle includes two different approaches: anion exchange and cation exchange according to the charge of the ligands on the ion exchange resin. A conventional IEC purification process usually consists of one or more: equilibration sections, application or loading sections, wash sections, elution sections, and regeneration sections (cf. Remington's Pharmaceutical Sciences, Gennaro, ed., Mack Publishing Co., Easton, Pa., 1990, or Remington: The Science and Practice of Pharmacy, 19th Edition (1995)).

U.S. Pat. No. 6,451,987 discloses an ion exchange chromatography process for purifying a peptide from a mixture containing the peptide and related impurities.

U.S. Pat. No. 7,276,590 discloses an ion exchange chromatography process for purifying a peptide from a mixture containing the peptide and related impurities.

The cost of preparative systems has escalated tremendously. Furthermore, such systems operating at these high pressures are being used in routine production environment, which, can be a serious hazard. The industry is thus in the position of having to choose between low pressure, low cost systems which are slow and have limited purification capabilities, or high pressure, high performance systems which are significantly more expensive and pose a health hazard. Additionally, biologicals may degrade in time while in the preparative solution, either thermally or due to the presence of proteases and the like, so speedy separation is very desirable. The efficiency of production achieved with a liquid chromatographic separation process for biological macromolecules can be described in terms of amount-of-product/dollar. To achieve optimum production, both production speed and capacity are important considerations that are currently not well met. Thus, a need exists in the art of chromatography for a system which is capable of high performance without resorting to high pressure and the associated hazards and high costs.

This need would be satisfied when the process duplicates as much as possible the yield, purity, throughput, and operating conditions of the chromatographic process wherein elution is conducted by selected solvent system, pH range and other related factors. Operating procedures may be advantageously employed for commercial separations. Efforts have been made to create Ion-Exchange/HPLC systems in which separations are characterized by higher resolutions.

Thus, when used either alone or in combination with standard extraction and chromatographic techniques, the extractive methods of the invention allow for the isolation of molecules of the subject invention in high yield and high purity with fewer steps than are required by conventional methods.

The object of the present invention is therefore to provide a preparative chromatography system for efficient separation and purification with a small scale and low cost, without the drawbacks of conventional separation and purification systems.

More particularly, the present invention relates to methods for conducting very high efficiency chromatographic separations, i.e., chromatography techniques characterized by both high resolution and high throughput per unit volume of chromatography matrix. More specifically, the invention relates to particularly preparative chromatography, and to methods for conducting chromatographic separations at efficiencies hereto for unachieved.

Additional objects, advantages and novel features of the invention, together with additional features contributing thereto and advantages accruing there from will be apparent to those skilled in the art, from the following description of the invention which is shown in the accompanying drawings which are incorporated herein by reference and form an integral part hereof. The objects and advantages of the invention may be realized and attained by means of the instrumentalities and combinations particularly pointed out in the appended claims.

### SUMMARY OF THE INVENTION

Accordingly, the present invention relates to a preparative chromatographic method for purifying a polypeptide from a mixture containing at least one related impurity, said method comprising steps of a) subjecting the polypeptide mixture to chromatographic process wherein the resin is washed and equilibrated in a buffer and an organic modifier at slightly acidic pH, b) eluting by a convex or a concave non-linear gradient of steepness having steepness coefficient ranging from zero to co running from buffer concentrations in the range of 0.01 M to 1 M; and c) recovering the polypeptide devoid of target impurities by at least 50%; purified IN-105 according to the method above with a purity of at least 95%; a method of purifying insulin and analog from mixture containing at least one related impurity, said method comprising steps of a) subjecting the mixture to RPHPLC column which is washed and equilibrated with organic modifier, b) eluting by a convex/concave non linear gradient of steepness having steepness coefficient ranging from zero to co running from buffer concentrations in the range of 0.01 M to 1 M and c) recovering insulin and analog devoid of target impurities by at least by 50%; purified Insulin methyl ester according to any of the methods above with purity of at least 85%; purified Insulin methyl ester according to any of the methods above with purity of at least 90%; purified glargine according to any of the methods above with purity of at least 90%; purified glargine according to any of the methods above with purity of at least 95%; purified aspart according to any of the methods above with purity of at least 80%; purified aspart according to any of the methods above with purity of at least 84%; and purified aspart according to any of the methods above with purity of at least 88%.

### DETAILED DESCRIPTION OF THE INVENTION

The present invention relates to a preparative chromatographic method for purifying a polypeptide from a mixture containing at least one related impurity, said method comprising steps of:
   (a) subjecting the polypeptide mixture to chromatographic process wherein the resin is washed and equilibrated in a buffer and an organic modifier at slightly acidic pH;
   (b) eluting by a convex or a concave non-linear gradient of steepness having steepness coefficient ranging from zero to ∞ running from buffer concentrations in the range of 0.01 M to 1 M; and
   (c) recovering the polypeptide devoid of target impurities by at least 50%.

In an embodiment of the present invention, the chromatographic process is selected from a group comprising Ion Exchange Chromatography or Reverse Phase-HPLC or a combination thereof.

In another embodiment of the present invention, the resin in step a) is an ion exchange resin or C4 to C18 silica resin.

In yet another embodiment of the present invention, the buffer employed is an acetate buffer.

In still another embodiment of the present invention, the pH ranges from about 2 to about 5.

In still another embodiment of the present invention, the polypeptide is Insulin, Insulin analog or a derivative thereof.

In still another embodiment of the present invention, the insulin derivative is IN 105.

In still another embodiment of the present invention, the Insulin analog is glargine, aspart, lispro, glulisine or Insulin methyl ester.

In still another embodiment of the present invention, the target impurity is des-Octa impurity, des thero impurity, des octa aspart impurity or flank and monoglycosylated aspart or any combination thereof.

In still another embodiment of the present invention, the ratio of the polypeptide to the resin on a weight by volume ratio is in the range from 0.1 to 50 g/l.

In still another embodiment of the present invention, the ratio of the polypeptide to the resin on a weight by volume ratio is in the range more preferably 1 to 25 g/l.

In still another embodiment of the present invention, the recovered polypeptide has a purity of at least 95%.

The present invention relates to purified IN-105 according to any of the preceding embodiments with a purity of at least 95%.

The present invention relates to a method of purifying insulin and analog from mixture containing at least one related impurity, said method comprising steps of:
(a) subjecting the mixture to RPHPLC column which is washed and equilibrated with organic modifier.
(b) eluting by a convex/concave non linear gradient of steepness having steepness coefficient ranging from zero to co running from buffer concentrations in the range of 0.01 M to 1 M; and
(c) recovering insulin and analog devoid of target impurities by at least by 50%

In an embodiment of the present invention, the RPHPLC is washed and equilibrated with pH ranging from about 2 to about 5.

In another embodiment of the present invention, the recovered insulin analog has a purity of at least 97%.

The present invention relates to purified Insulin methyl ester according to any of the preceding embodiments with purity of at least 85% to 90%

The present invention further relates to purified Glargine according to any of the preceding embodiments with purity of at least 90% to 95%.

The present invention relates to purified Aspart according to any of the preceding embodiments with purity of at least 80% to 88%.

A process for the purification of an impure preparation containing IN-105 by means of an ion-exchange and/or reverse phase preparative chromatography process is provided. In an illustrative embodiment a chromatographic column is loaded with a stationary phase, typically a ceramic based resin. The typical process includes feeding a crude IN-105 solution into the chromatographic column, applying a mobile phase to the chromatographic column, and recovering the IN-105 eluate from the chromatographic column. Each separated eluate containing the target compound however, has sufficient recovery and purity.

A preparative chromatographic method for purifying a polypeptide from a mixture containing at least one related impurity, wherein said method comprises
(a) Applying the polypeptide mixture to an ion-exchange resin which is washed and equilibrated in a buffer at slightly acidic pH.
(b) Elution employing a convex/concave non-linear gradient of steepness in the range zero to co running from buffer concentrations in the range of 0.01M to 1 M.
(c) Recovering the polypeptide devoid of target impurities by at least 50%.

More particularly, the highlight of the invention relates to elution employing a convex/concave non-linear gradient of steepness in the range zero to ∞ going from buffer concentrations in the range of 0.01 M to 1 M resulting in the reduction of target impurities in the range of 60-95%. The process thus affords purity of the target compound in the range of at least 80%-97%.

A process for the purification of impure preparation containing insulin and analogs by RP HPLC, wherein said method comprises
a) RPHPLC column is first equilibrated with right percentage of organic modifier.
b) The protein sample is injected onto the column under overloaded column condition.
c) Wash the column with certain percentage of organic modifier in order to remove any unbound proteins.
d) Followed by gradient elution which has been carried out under non-linear conditions.
e) Samples are fractionated and analysed to estimate overall purity.
f) Fractions meeting require purity are pooled together to give an elution pool and column is then regenerated to remove any proteins that may be tightly bound.

While the present invention has been described with reference to the specific embodiments thereof, it should be understood by those skilled in the art that various changes may be made and equivalents may be substituted without departing from the true spirit and scope of the invention.

In this specification the nature and theoretical underpinnings of the required operational parameters of preparative chromatography is first disclosed, followed by equational principles useful in optimization of the chromatographic processes to specific instances, disclosure of specific materials that are useful in the practice of such chromatography, and specific examples of such procedures.

An ordinary artisan should require no additional explanation in developing the methods and systems described herein but may nevertheless find some helpful guidance in the preparation of these methods and systems by examining standard reference works in the relevant art. For example, an ordinary artisan may choose to review L. R. Snyder, “Gradient Elution”, from High Performance Liquid Chromatography, Cs. Horvath (ed.), Academic Press, 1980, pp. 207-316 and M. A. Stadalius, H. S. Gold and L. R. Snyder, J. Chromatography, 296 (1984), 31-59, Cazes, J., 2005, “Encyclopedia of Chromatography” (pp 718). Marcel Dekker, Edition 2, Vol 1. the disclosures of which are hereby incorporated by reference in their entirety.

### DEFINITION OF TERMS

The term “ion-exchange” and “ion-exchange chromatography” refers to the chromatographic process in which a solute of interest (such as a protein) in a mixture interacts with a charged compound linked (such as by covalent attachment) to a solid phase ion exchange material such that the solute of interest interacts non-specifically with the charged compound more or less than solute impurities or contaminants in the mixture. The contaminating solutes in the mixture elute from a column of the ion exchange material faster or slower than the solute of interest or are bound to or excluded from the resin relative to the solute of interest. “Ion-exchange chromatography” specifically includes cation exchange, anion exchange, and mixed mode chromatography.

The term “preparative chromatographic separation” is intended to mean a chromatographic separation for isolating or separating a component or components (e.g., a desired component) of a chemical mixture. A preparative chromatographic separation can comprise eluting one or more components through a preparative column. A preparative chromatographic separation can be characterized and/or affected by preparative chromatographic parameters.

The phrase “gradient mode of chromatography” herein refers to a flowing solvent composition that changes as a function of time, typically in response to a user defined profile. A solvent has a composition which may, for example, a mixture of two solutions, referred to herein as mobile-phase Buffer A and mobile-phase Buffer B. In preferred aspects, the column is a strong anionic or strong cationic exchange column, which means that the column adsorbent's ion exchange properties do not change over the working pH range of the column.

A gradient chromatography system uses the same general components as the isocratic system, the primary difference being in the solvent delivery which has to deliver a mixture of fluids whose composition varies continuously as a function of time. The gradient technique is often used to separate peaks that may elute close together in an isocratic mode and need small changes in elution conditions to achieve differential separation.

Gradient elution is performed by changing from a weak to a strong eluent during a chromatography run. In gradient elution, the elution process begins with an eluent of low displacing power than increases over time to an eluent of greater displacing power. This can be accomplished by changing the concentration and/or composition of the eluent. The flow rates used for the described system are typical for ion chromatography. Gradient elution is defined as elution performed by changing from a weak to a strong eluent during a run. Such an eluent is referred to as a gradient eluent. Examples of suitable gradient eluents are illustrated in Rocklin, R. D. et al (Journal of Chromatography 411 (1987) 107.) and in Ion Chromatography, Small, H. (Plenum Press 1989) pp. 187, 213. They include increases in eluent strength as a function of time in the shape of linear, concave, convex, step, linear with hold periods, and combinations of these functions.

Cation exchange chromatography helps in separation of proteins from its impurities based on their charge difference. In most of the CIEX runs, elution is carried out in isocratic gradient mode having same concentration of buffer throughout. Applying a non-linear gradient during elution helps in separation of closely related impurities because of the gradual change in the buffer concentration with time. Furthermore the application of a nonlinear gradient results in either a sharp increase in concentration followed by a gradual rise in the concentration or a gradual increase in concentration followed by a sharp rise in the concentration. This non linear variation helps improve separation of closely eluting impurities.

The external gradient is generated by the continuous mixing of a starting buffer with a second buffer at a different concentration, simultaneously increasing the proportion of the second buffer until the mixture reaches the desired concentration. Each of the concentration gradient forming buffer solutions can consist of either the same buffering components or different buffering components.

As such, the gradient can be a “linear gradient,” a “convex gradient,” a “concave gradient,” or a “discontinuous gradient,” or any other suitable form known to the skilled artisan.

“Convex gradient” refers to a gradient wherein the buffer concentration varies gradually over the initial phase with a sharp rise later.

“Concave gradient” refers to a gradient wherein the buffer concentration has a sharp increase in the slope initially followed by a gradual rise later.

“Resolution” is a measure of the degree of purification that a system can achieve. This variable is controlled by the nature of solutes in the process liquor and the chemical properties of the interactive surface of the chromatography medium and also characteristics that affect the dynamics of flow, diffusion and sorption kinetics.

The term ‘polypeptide’, ‘protein’, ‘peptide’ refers to a polymer of amino acids and does not refer to a specific length of the product; thus, peptides, oligopeptides, and proteins are included within the definition of polypeptide. This term also does not refer to or exclude post-expression modifications of the polypeptide although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. In one embodiment, the molecule is a polypeptide or their related analogs or derivatives thereof.

The term “purifying” a protein from a composition comprising the protein and one or more contaminants thereby increasing the degree of purity of protein in the composition by reducing the contents of at least one contaminant from the protein composition.

An “impurity” is a material that is different from the desired polypeptide product or protein of interest. The main impurity targeted in cation-exchange chromatography using non-linear gradients has been des-Octa IN-105. In enzymatic reactions, trypsin converts insulin precursor into insulin by cleaving the leader and linker sequences. During this reaction, one of the impurities that gets generated is des-Octa IN-105 in which the trypsin action happens at 8 amino acids away from the C-terminal of B chain. The relative retention of this impurity on an octadecyl ceramic column with respect to the retention of the product is 0.84. The impurity thus described is referred to as the “target impurity”.

The term “Insulin analog” is intended to encompass any form of “Insulin” as defined above wherein one or more of the amino acids within the polypeptide chain has been replaced with an alternative amino acid and/or wherein one or more of the amino acid has been deleted or wherein one or more additional amino acids has been added to the polypeptide chain. Insulin analog is selected from the group comprising Aspart, Glargine and Insulin methyl ester.

‘IN-105’ is an insulin molecule conjugated at the epsilon amino acid Lysine at position B29 of the insulin B-chain with an ampiphilic oligomer of structural formula CH3O—(C4H2O)3—CH2—CH2—COOH. The molecule may be monoconjugated at A1, B1 and B29, di-conjugated at various combinations of A1, B1 and B29, or triconjugated at various combinations of A1, B1 and B29.

The main impurities targeted in RPHPLC using non linear gradient for insulin and analog comprises:

Des-Threo Insulin—In enzymatic reaction, trypsin converts insulin precursor into insulin by cleaving the leader and linker sequences. During this reaction, one of the impurities that gets generated is des-threo in which the trypsin action happens at the 29^{th} amino acid of the B chain. The relative retention of this impurity on an octadecyl silica column with respect to the retention of the product is 0.98.

Des-Octa Aspart—In enzymatic reaction, trypsin converts insulin aspart precursor into insulin aspart by cleaving the leader and linker sequences. During this reaction, one of the impurities that gets generated is des-octa in which the trypsin cleaves at 8 amino acids away from the C-terminal of the B chain. The relative retention of this impurity on an octadecyl silica column with respect to the retention of the product is 0.9.

Flank: In aspart this is also one of the major impurities seen. In enzymatic reaction, trypsin converts insulin aspart precursor into insulin aspart by cleaving the leader and linker sequences. During this reaction, one of the impurities that gets generated is flank, which happens when the C-peptide (linker peptide) does not cleave completely. The relative retention of this impurity on an octadecyl silica column with respect to the retention of the product is 0.96.

Glycosylated Aspart: The host is known to carry out post-translational modification resulting in the addition of mannosyl groups to the precursor. Correspondingly the impurity generated is glycosylated form of the product which is also known as glycosylated impurity. The relative retention of this impurity on an octadecyl silica column with respect to the retention of the product is 0.91.

Steepness co-efficient refers to the slope of the concentration curve with respect to time. Some embodiments of the invention may employ a nonlinear solvent strength gradient (e.g., a piecewise linear solvent strength gradient, a concave gradient shape, or a convex gradient shape) for either or both analytical and preparative chromatographic separations.

Some embodiments of the invention may comprise first optimizing or improving certain aspects of an analytical chromatographic separation (e.g., by selecting an appropriate value for the analytical gradient steepness parameter) prior to optimizing and or improving separation of a desired component using a preparative chromatographic separation. A steep gradient is typically efficient for a continuous flow operation.

A typical example of a method for the separation and purification of proteins of the invention comprises of the following steps:
i. Packing the Ion-exchange column with a ceramic based resin equilibrated with about 10mM of an acetate buffer.
ii. Loading the peptide composition containing at least one related impurity on the column at a flow rate of about ≦100-400 cm/hr.
iii. Eluting the purified product from the column performing a non-linear gradient employing a convex/concave gradient of steepness in the range zero to co going from buffer concentrations in the range of 0.01 M to 1 M.

The pH of the elution buffer may be from about 2 to about 9, alternatively from about 3 to about 8, from about 4 to about 8, or from about 5 to about 8, although the pH or pH range for elution will be determined according to the desired protein of interest and the type of chromatography practiced. Appropriate pH ranges for a loading, wash, or elution buffer are readily determined by standard methods such that the protein of interest is recovered in an active form.

In one embodiment of the disclosure, the aqueous buffer system is selected from sodium acetate buffer systems. In another aspect, an acetic acid aqueous buffer system may also be paired with one or more of ammonium acetate as salt/ion exchange compounds. In a further aspect, sodium hydroxide may optionally be used to adjust pH or sodium ion concentration. In a preferred aspect, the ion exchange buffer/buffer salt ion exchange compound in the mobile phase is an Ammonium acetate/sodium acetate buffer system.

Another embodiment of the invention relates to the separation gradient formulae, equations (i) and (ii) can be used for the calculation of the non-linear gradients. A gradient is said to be linear if the change in the solvent concentration with time is linear. Non linear gradients can be calculated by the following formula. If θ is the fraction of the solvent at any time t, C is the solvent concentration at that time, C1 the concentration at the start of the gradient, C2 the concentration at the end of the gradient, and tg the total time of the gradient, then
(i) Concave gradient−θ=(t/tg)̂n; C=(C2−C1)*θ+C1
(ii) Convex gradient−θ=1−(1−t/tg)̂n; C=(C2−C1)*θ+C1
   Where n is the steepness coefficient (n=zero to ∞).

The invention features, in another aspect, a method of purifying a protein, the method including the steps of subjecting the protein mixture containing at least one related impurity that includes loading the protein mixture onto a Ion-exchange column containing an ion-exchange based ceramic resin, equilibrated with a suitable buffer at slightly acidic pH, subsequent elution employing a convex/concave gradient of steepness in the range zero to co going from buffer concentrations in the range of 0.01 M to 1 M resulting in the reduction of target impurities in the range of 60-75%. The eluted protein can be further subjected to high-performance liquid chromatography for further processing.

In another aspect, the invention features a method of purifying Insulin and analog by carrying out RP HPLC. The method includes equilibrating the RP HPLC column with the right percentage of organic modifier, thereafter sample is injected onto the column under overloaded column conditions, then the column is washed with certain percentage of organic modifier in order to remove the any unbound protein, followed by gradient elution, which has been carried out under no-linear condition in the present examples. Further the samples are fractionated and analysed to estimate overall purity and fractions meeting the specifications for required purity are pooles together to give an elution pool.

According to one aspect of the invention the purified protein product is devoid of or contains substantially minimal amounts of target impurities. According to one aspect of the invention the purity of the purified protein product is at least 95%, according to another aspect of the invention the purity of the purified protein product is at least 97%, according to yet another aspect of the invention the purity of the purified protein product is at least 98%, according to yet another aspect of the invention the purity of the purified protein product is at least 99%, according to still another aspect of the invention the purity of the purified protein product is 100%.

Accordingly to another aspect of the invention the purified insulin and analog is devoid of or contains substantially minimal amounts of target impurities. According to one aspect of the invention the purity of purified insulin and analogs is at least 80%, accordingly to another aspect of the invention the purity of the purified insulin and analog is at least 85%, accordingly another aspect of the invention the purity of the purified insulin and analog is at least 90%, according to yet another aspect of the invention the purity of the insulin and analog is 97%.

Another aspect of the invention relates to an effective increase in protein purity (>95%) by carrying out reverse phase chromatography in combination with ion-exchange chromatography carried out using non-linear gradient.

These and other non-limiting embodiments of the present invention are readily understood by one of ordinary skill in the art upon reading the disclosure and claims provided herein. It is understood that this invention is not limited to the particular methods and processes described, as such desired protein/peptide products and methods may, of course, vary. It is also to be understood that the terminology used herein is for the purpose of describing particular embodiments only, and is not intended to be limiting.

It will be appreciated that the use of the exemplified method of purification as described in the Examples to obtain high resolution separations, coupled with the components used for separation, makes it especially efficient for obtaining the desired peptide in a particularly simple, convenient and inexpensive manner.

The foregoing descriptions of specific embodiments of the present invention are presented for purposes of illustration and description. They are not intended to be exhaustive or to limit the invention to the precise forms disclosed. Various modifications and variations are possible in view of the above teachings. In addition, many modifications may be made to adapt a particular situation, material, composition of matter, process, process step or steps, to the objective, spirit and scope of the present invention. All such modifications are intended to be within the scope of the claims appended hereto.

The technology of the instant Application is further elaborated with the help of following examples. However, the examples should not be construed to limit the scope of the invention.

### EXAMPLE 1

Crude IN-105 was diluted with water 5 times and pH was adjusted to 3.5. This was loaded onto a steel column packed with Ceramic Hyper-D S cation exchange resin. The equilibration and wash was carried out with 10 mM Ammonium acetate buffer at pH 3.5. The load purity was 46.22% containing 5.6% des-Octa impurity. When a convex gradient of steepness 0.3 was employed going from a buffer concentration of 0.2 M to 0.64 M of Ammonium acetate over 10 column volumes, the resulting elution pool showed a purity of 59% with desocta levels at 4.4% only, indicating an approximate reduction in des-Octa impurity levels by 50%.

### EXAMPLE 2

Crude IN-105 was diluted with water 5 times and pH was adjusted to 3.5. This was loaded onto a steel column packed with Ceramic Hyper-D S cation exchange resin. The equilibration and wash was carried out with 10 mM Ammonium acetate buffer at pH 3.5. The load purity was 52.57% containing 9.1% des-Octa impurity. When a concave gradient of steepness 0.15 was employed going from a buffer concentration of 0.38 M to 0.64 M of Ammonium acetate over 10 column volumes, the resulting elution pool showed a purity of 63.31% with desocta levels at 5.14% only, indicating an approximate reduction in des-Octa impurity levels by 71%.

### EXAMPLE 3

Crude IN-105 was diluted with water 5 times and pH was adjusted to 3.5. This was loaded onto a steel column packed with Ceramic Hyper-D S cation exchange resin. The equilibration and wash was carried out with 10 mM Ammonium acetate buffer at pH 3.5. The load purity was 55.46% containing 8.18% des-Octa impurity. When a concave gradient of steepness 0.15 was employed going from a buffer concentration of 0.2 M to 0.64 M of Ammonium acetate over 10 column volumes, the resulting elution pool showed a purity of 67.37% with desocta levels at 5.35% only, indicating an approximate reduction in des-Octa impurity levels by 61%.

### EXAMPLE 4

Crude IN-105 was diluted with water 5 times and pH was adjusted to 3.5. This was loaded onto a steel column packed with Ceramic Hyper-D S cation exchange resin. The equilibration and wash was carried out with 10 mM Ammonium acetate buffer at pH 3.5. The load purity was 55.80% containing 7.71% des-Octa impurity. When a convex gradient of steepness 0.15 was employed going from a buffer concentration of 0.38 M to 0.64 M of _Ammonium acetate over 10 column volumes, the resulting elution pool showed a purity of 70% with desocta levels at 3.85% only, indicating an approximate reduction in des-Octa impurity levels by 75%.

### EXAMPLE 5

Crude IN-105 was diluted with water 5 times and pH was adjusted to 3.5. This was loaded onto a steel column packed with Ceramic Hyper-D S cation exchange resin. The equilibration and wash was carried out with 10 mM Ammonium acetate buffer at pH 3.5. The load purity was 55.99% containing 8.15% des-Octa impurity. When a convex gradient of steepness 0.15 was employed going from a buffer concentration of 0.38 M to 0.64 M of Ammonium acetate over 20 column volumes, the resulting elution pool showed a purity of 70.32% with desocta levels at 3.91% only, indicating an approximate reduction in des-Octa impurity levels by 74%.

### EXAMPLE 6

Crude IN-105 was diluted with water 5 times and pH was adjusted to 3.5. This was loaded onto a steel column packed with Ceramic Hyper-D S cation exchange resin. The equilibration and wash was carried out with 10 mM Ammonium acetate buffer at pH 3.5. The load purity was 53.51% containing 9.29% des-Octa impurity. When a convex gradient of steepness 0.15 was employed going from a buffer concentration of 0.38 M to 0.64 M of Ammonium acetate over 20 column volumes, the resulting elution pool showed a purity of 68.41% with desocta levels at 4.79% only, indicating an approximate reduction in des-Octa impurity levels by 75%.

### EXAMPLE 7

Crude IN-105 was loaded onto a steel column packed with Ceramic Hyper-D S cation exchange resin and a convex gradient was run. The Elution pool obtained from the cation exchange was diluted and loaded on to Silica C8 resin containing 10% solvent. The mobile phase used was 0.25 M Citric acid (Buffer A) and acetonitrile (Buffer B). Equilibration was carried out at 10% B and wash was at 15% B. The load purity was 78.26%. Elution was carried out at a linear gradient of 15% B to 23% B over 20 column volumes, the resulting elution pool showed a purity of 96.33%.

### EXAMPLE 8

Crude IN-105 was loaded onto a steel column packed with Ceramic Hyper-D S cation exchange resin and a convex gradient was run. The Elution pool obtained from the cation exchange was diluted and loaded on to Silica C8 resin containing 10% solvent. The mobile phase used was 0.1 M Tris and 0.02 M MgCl₂ (Buffer A) and acetonitrile (Buffer B). Equilibration and wash were carried out at 10% B. The load purity was 65.06%. Elution was carried out at a linear gradient of 25% B to 32% B over 20 column volumes, the resulting elution pool showed a purity of 92.87%.

### EXAMPLE 9

Post TP insulin methyl ester (IME) crystals were dissolved in 0.5 N HOAc, 50 mM Na-acetate and 15% MeOH such that the concentration of the load was 1.5 gpl. This was then filtered and loaded on a C8 silica based RP column at 20 gpl (i.e. 20 g of IME per litre of resin). The load purity was 74.51% containing approximately 9% DesThreo human insulin as the major impurity. The RP HPLC was carried out using Sodium acetate buffer (Buffer A) and Methanol (Buffer B). A convex gradient from 50-65% B with a steepness of 3 was used and the resulting product contained 0% DesThreo human insulin and had an overall purity of 95.95%

### EXAMPLE 10 a)

Glargine crystals post enzyme reaction were dissolved in 2 M HOAc and 10% acetonitrile such that the load concentration was between 0.8 to 0.95 gpl. The load was then filtered and loaded onto a C18 silica based RP column at a loading capacity of 5.4 gpl (i.e. 5.4 g of insulin glargine per litre of resin). The load purity was between 35%-40% containing several different impurities. Elution was carried out using 250 mM Citric acid (Buffer A) and IPA (Buffer B) as the organic modifier. The final EP purity was 96.02% with the isolation of different impurities. For an overall purity of 80.97% step yield increase to 86.7% A convex gradient from 10-20% B with a steepness of 0.5 was used.

### EXAMPLE 10 b)

Glargine crystals post enzyme reaction were dissolved in 2 M HOAc and 10% acetonitrile such that the load concentration was between 0.8 to 0.95 gpl. The load was then filtered and loaded onto a C18 silica based RP column at a loading capacity of 5.4 gpl (i.e. 5.4 g of insulin glargine per litre of resin). The load purity was between 35%-40% containing several different impurities. Elution was carried out using 250 mM Citric acid (Buffer A) and IPA (Buffer B) as the organic modifier. The final EP purity was 96.02% with the isolation of different impurities. For an overall purity of 80.97% step yield increase to 86.7% A concave gradient from 10-20% B with a steepness of 0.5 was used.

### EXAMPLE 11

Enzyme end material of insulin aspart was diluted with acetic acid and acetonitrile such that the load concentration was up to 0.8 gpl. The load was then filtered and loaded onto a C4 silica based RP column at a loading capacity of 10 gpl (i.e. 10 g of insulin aspart per litre of resin). The load purity was 71.03% containing several different closely related impurities such as des-octa aspart, flank, monoglycosylated aspart, etc. Elution was carried out using 25 mM sodium acetate at pH 4.0 (Buffer A) and acetonitrile (Buffer B). A concave gradient from 15-30% B with a steepness of 0.5 was used and the resulting product had an overall purity of 88.24%. The major advantage of employing the non-linear gradient was the reduction in the impurities such as flank (reduced by 97%) and des-octa aspart (reduced by 70%).

Preferred embodiments of the present invention have been disclosed. A person of ordinary skill in the art would realize, however, that certain modifications would come within the teachings of this invention. Therefore, the following claims should be studied to determine the true scope and content of the invention.

We claim:

1. A preparative chromatographic method for purifying a polypeptide from a mixture containing at least one related impurity, said method comprising steps of:
(a) subjecting the polypeptide mixture to chromatographic process wherein the resin is washed and equilibrated in a buffer and an organic modifier at slightly acidic pH;
(b) eluting by a convex or a concave non-linear gradient of steepness having steepness coefficient ranging from zero to ∞ running from buffer concentrations in the range of 0.01 M to 1 M; and
(c) recovering the polypeptide devoid of target impurities by at least 50%.

2. The method according to claim 1, wherein the chromatographic process is selected from a group comprising Ion Exchange Chromatography or Reverse Phase-HPLC or a combination thereof.

3. The method according to claim 1, wherein the resin in step a) is an ion exchange resin or C4 to C18 silica resin.

4. The method according to claim 1, wherein the buffer employed is an acetate buffer.

5. The method according to claim 1, wherein the pH ranges from about 2 to about 5.

6. The method according to claim 1, wherein the polypeptide is Insulin, Insulin analog or a derivative thereof.

7. The method according to claim 6, wherein insulin derivative is IN 105.

8. The method according to claim 6, wherein Insulin analog is glargine, aspart, lispro, glulisine or Insulin methyl ester

9. The method according to claim 1, wherein the target impurity is des-Octa impurity, des thero impurity, des octa aspart impurity or flank and monoglycosylated aspart or any combination thereof.

10. The method according to claim 1, wherein the ratio of the polypeptide to the resin on a weight by volume ratio is in the range from 0.1 to 50 g/l.

11. The method according to claim 1, wherein the ratio of the polypeptide to the resin on a weight by volume ratio is in the range more preferably 1 to 25 g/l.

12. The method according to claims 1 to 11, wherein the recovered polypeptide has a purity of at least 95%.

13. Purified IN-105 according to any of the preceding claims with a purity of at least 95%

14. A method of purifying insulin and analog from mixture containing at least one related impurity, said method comprising steps of:
(a) subjecting the mixture to RPHPLC column which is washed and equilibrated with organic modifier.
(b) eluting by a convex/concave non linear gradient of steepness having steepness coefficient ranging from zero to ∞ running from buffer concentrations in the range of 0.01 M to 1 M ; and
(c) recovering insulin and analog devoid of target impurities by at least by 50%

15. The method according to claim 14, wherein the RPHPLC is washed and equilibrated with pH ranging from about 2 to about 5.

16. The method according to claim 14, wherein the recovered insulin analog has a purity of at least 97%.

17. Purified Insulin methyl ester according to any of the preceding claims with purity of at least 85%

18. Purified Insulin methyl ester according to any of the preceding claims with purity of at least 90%.

19. Purified glargine according to any of the preceding claims with purity of at least 90%.

20. Purified glargine according to any of the preceding claim with purity of at least 95%.

21. Purified aspart according to any of the preceding claim with purity of at least 80%

22. Purified aspart according to any of the preceding claim with purity of at least 84%

23. Purified aspart according to any of the preceding claim with purity of at least 88%
